(19) United States
(12) Patent Application Publication
(10) Pub. No.: US 2018/0078516 A1
(43) Pub. Date: Mar. 22, 2018

(54) Pharmaceutical Composition of Ibuprofen Sodium for Oral Administration

(71) Applicant: InnovaZone Labs LLC, Sunrise, FL (US)

(72) Inventors: Julio César Villamil Torres, Bogota (CO); Nuria Sanz Menéndez, Madrid (ES); Angel Muñoz Ruiz, Madrid (ES)

(21) Appl. No.: 15/269,910

(22) Filed: Sep. 19, 2016

(51) Int. Cl. A61K 31/192; A61K 9/08

(52) U.S. Cl. CPC A61K 31/192; A61K 9/08

(57) ABSTRACT

The present invention relates to a pharmaceutical composition of ibuprofen sodium in the form of oral solution, comprising a taste masking system based on sodium bicarbonate and povidone. The combination of the masking effect of chemical type facilitated by sodium bicarbonate, with the masking effect of physical type favored by povidone, completely eliminates the bitter taste and the feeling of irritation and burning perceived in the throat when ibuprofen is administered in soluble form. This invention also relates to the use of the referred composition for the treatment of pain, inflammation, and/or fever, particularly in pediatric patients.

### FIELD OF THE INVENTION

The present invention relates to a pharmaceutical composition and methods of use thereof, more specifically to a pharmaceutical composition comprising ibuprofen sodium as aqueous solution for oral administration, which is particularly suitable for pediatric administration.

### BACKGROUND

Ibuprofen is a drug belonging to the group of non/steroidal anti-inflammatory drugs, commonly called NSAIDs, specifically to the family of propionic acid derivatives, possessing analgesic, antipyretic, and anti-inflammatory actions. Ibuprofen was originally described in 1960 in a group of related patents of the company Boots Pure Drug Co. Ltd. (for instance, in the British Patent GB971700 or the US equivalent patent U.S. Pat. No. 3385886), and was developed as a better alternative to aspirin. Pharmacologically, ibuprofen acts as a non-selective inhibitor of cyclooxygenase 1 (COX-1) and cyclooxygenase 2 (COX-2), and this activity is the one responsible for its therapeutic effect, since cyclooxygenases are involved in the synthesis of prostaglandins, which in turn play a very important role in the processes of pain, inflammation, and fever. Ibuprofen is the most commonly used NSAID and the most frequently prescribed by physicians worldwide. Ibuprofen is prescribed in patients of all ages, and it's also widely used in pediatrics for the treatment of pain, inflammation, and/or fever from 3 months of age. Ibuprofen is a relatively weak acid with a pKa value of 5.3; it's practically insoluble in water, and has a characteristic bitter taste. It belongs to the class II of the biopharmaceutical classification system, meaning that it has a very low solubility, but very high permeability. After oral administration, the bioavailability of ibuprofen compared with that of an equivalent dose administered intravenously, is close to 100%, reaching the peak plasma concentration between 1 and 2 hours after administration. Thus, while the bioavailability of ibuprofen is high, its low solubility makes the drug reach the bloodstream relatively slowly, so its therapeutic effects begins between 45 minutes and one hour after oral administration.

This slow onset of action is inconvenient, especially considering that the administration of any NSAID, particularly in the pediatric population, is expected to obtain fast effects in reducing pain, inflammation, and/or fever.

The use of ibuprofen for the pediatric population also involves a particular problem, as its dosage should be carefully adjusted to the weight of each child. The dosage is usually a total daily dose of 20 to 30 mg/kg, divided in three or four doses. In addition, ibuprofen for pediatric patients should be formulated in a way that is organoleptically acceptable and easily swallowed by the child patient.

That is why ibuprofen oral formulations in liquid form are common, usually with an ibuprofen concentration between 2% and 4%, which are also available for patients with zo convenient graduated devices or droppers to administer variable doses depending on the patient weight.

However, the low solubility of ibuprofen and its bitter taste, together with its relatively slow absorption, make their liquid pediatric formulations for oral administration problematic.

In the state of the art, several ibuprofen presentations containing the active ingredient in its acid, insoluble form, as aqueous suspensions for oral administration are described. Thus, for example, in the European patent application EP0257288-A1, an aqueous suspension containing from 50 to 400 mg of ibuprofen per 5 mL, more than 50% by weight of polyalcohol, a suspending agent, sodium hydroxide or potassium to adjust the pH to a value between 7.0 and 7.7, an antioxidant, and sweeteners is described. The compositions preferably have a very sweet flavor to mask the unpleasant taste of ibuprofen.

In the European patent application EP0298740-A1, a pediatric ibuprofen composition in which the active ingredient is maintained suspended by using a mixture of suspending agents, particularly xanthan gum, cellulose microcrystalline, sodium carboxymethylcellulose, and polysorbate 80 is described. The composition contains between 55% and 75% by weight of a combination of flavor masking agents, including sucrose and sorbitol solution, plus citric acid in an amount sufficient to adjust the pH to a value between 3.5 and 5.0.

In the European patent application EP0390369-A2, a pediatric composition based on ibuprofen and with characteristics similar to those referred in the above patent application is described, with the difference that a buffering system is used to maintain the pH of the preparation between 1.5 and 3.5, even when if it comes in contact with saliva, thus avoiding that part of ibuprofen from dissolving in the mouth and causing the unpleasant feeling of irritation and burning that ibuprofen typically generates in mouth and throat. Likewise, pharmaceutical compositions in which ibuprofen is suspended in a gel composed of polysaccharides or peptides, which, mask the typical unpleasant taste and sensation of irritation caused by this drug additionally are described in Japanese patent application JPH10182449.

These suspension formulations of ibuprofen, in which this drug is in its undissociated acid form, mask its bitter taste relatively effectively but the disadvantage of slow dissolution to reach the gastrointestinal tract persists. Therefore, the problem of a relatively long period of time for the onset of the therapeutic action is not solved.

In order to solve the problem of low speed with which ibuprofen is absorbed, different ibuprofen salts of high solubility have been used frequently. These salts decrease the time required to dissolve the active ingredient in the gastrointestinal tract, becoming available for absorption in significantly less time. Among the soluble salts of ibuprofen more widely studied are ibuprofen sodium, ibuprofen arginate, and ibuprofen lysinate. Nevertheless, the use of soluble salts of ibuprofen in pediatric pharmaceutical preparations for oral use creates new challenges from the organoleptic point of view, as both the bitter taste and the feeling of irritation and burning in the back part of the throat are persist when the salt is dissolved in its dissociated form within a pharmaceutical vehicle.

The problem of masking the flavor of aqueous solutions of ibuprofen soluble salts, in particular the ibuprofen sodium salt, is addressed in the European patent application EP0418043-A, wherein the use of weakly alkaline substances, specifically the bicarbonate salts, hydrogen-phosphate or alkali metal tribasic citrate, is suggested for masking the flavor. It indicates that to obtain an adequate masking effect, it is necessary to use an overage of the said masking agents in relation to the active ingredient, i.e., a significant excess of masking agents is necessary in order to successfully mask the taste of the active ingredient. The compositions described are powdery and typically supplied as one-dose package for reconstitution in water. Thus, the compositions described in the examples include an amount of ibuprofen salt corresponding to a dose of 200 or 400 mg of ibuprofen expressed as ibuprofen acid, together with the masking agent, a significant proportion of dextrose, as well as saccharin sodium and fragrances. The prepared compositions are reconstituted in 100 mL of water immediately before use.

However, this approach does not solve the problem found in pediatric use, since the final concentration of the active ingredient in the reconstituted solutions obtained is 0.2% or 0.4%, which are 10 times below the concentrations required for proper administration in children: 2% or 4%. To reach these concentrations, the amounts of bicarbonate salt, hydrogen-phosphate or tribasic citrate required to mask the unpleasant flavor of the active ingredient as described in the referred patent, would be very high, making sodium or potassium intake values undesirable and unsuitable for pediatric administration.

On the other hand, the compositions described in the patent application EP0418043-A are available in one-dose packages, wherein the entire contents of each package should be administered in a single dose; therefore, these compositions are not suitable for dose adjustments according to body weight of each patient, as required by pediatric administration.

The international patent application WO2013/072329-A1 describes a solid, non-effervescent, water soluble composition for reconstitution at the time of use, based on a soluble salt of ibuprofen, in which the unpleasant flavor of ibuprofen is masked by using a pharmaceutically acceptable strong base, which adjusts the pH of the preparation between 9.0 and 9.5. This composition is also available in one-dose packages.

The solution included in the referred patent is also not suitable for pediatric use, since the final concentration of the obtained solutions is very low in relation to the desired concentrations, and it does not allow adjustment of the dose. Besides, the flavor masking system based on preparing strong alkaline solutions is also not adequate for developing a ready-to-use pharmaceutical preparation, as the pH of 9.0 to 9.5 obtained at the time of reconstitution has the disadvantage of being incompatible with most preservative systems used in oral liquid pharmaceutical preparations.

Thus, a need remains for a pharmaceutical preparation based on a soluble salt of ibuprofen with a fast onset of action and that could be supplied as a ready-to-use solution, without requiring prior reconstitution, containing the adequate amounts of the active ingredient for proper administration in small volumes to pediatric patients, and masking the bitter taste and the feeling of irritation and burning in the mouth and throat caused by this active ingredient.

### SUMMARY OF THE INVENTION

The object of the present invention is a composition of ibuprofen sodium in an aqueous solution. In one preferred embodiment, a pharmaceutical composition for oral administration in the form of an aqueous solution comprises ibuprofen sodium dihydrate in a concentration ranging from 1.92% (w/v) to 6.41% (w/v), equivalent to 1.5% (w/v) to; 5.0% (w/v) of ibuprofen in acid form; povidone; and sodium bicarbonate. In one preferred embodiment, the ibuprofen sodium dihydrate concentration is 2.56% (w/v), equivalent to 2% (w/v) of ibuprofen in acid form. I another preferred embodiment, the ibuprofen sodium dihydrate concentration is 5.12% (w/v), equivalent to 4% (w/v) of ibuprofen in acid form.

In one further embodiment, the ratio of sodium bicarbonate to ibuprofen is between 1:1 and 1:5, wherein the amount of ibuprofen sodium is expressed as the equivalent amount of ibuprofen in its acid form. In another embodiment, the povidone is selected from the group consisting of povidone K-17, povidone K-30, and povidone K-90. In yet a further embodiment, the weight ratio of povidone to ibuprofen is between 1:0.4 and 1:8, wherein the amount of ibuprofen sodium is expressed as the equivalent amount of ibuprofen in its acid form. In another preferred embodiment, the composition has a pH value between 7.5 and 8.5.

In another embodiment of the present invention, the pharmaceutical composition also comprises a preservative agent. The preservative agent may be selected from the group consisting of methylparaben, ethylparaben, propylparaben, butylparaben, cetylpyridinium chloride, chlorhexidine acetate, chloramine T, and mixtures thereof. In a further embodiment, the preservative is a mixture of methylparaben and propylparaben.

In one further embodiment, the pharmaceutical composition includes a cosolvent. The cosolvent may be selected from the group consisting of propylene glycol, polyethylene glycol, sorbitol, ethanol, and mixtures thereof. In a further embodiment, the cosolvent is propylene glycol. In yet a further embodiment, the pharmaceutical composition also comprises a sequestering agent. The sequestering agent may be selected from the group consisting of edetic acid, disodium edetate, citric acid, fumaric acid, malic acid, and mixtures thereof. In some embodiments, the sequestering agent is disodium edetate. The pharmaceutical composition may further comprise at least one additional component selected from a sweetener, a flavor, and a crystallization inhibitor.

Another aspect of the invention is the use of said composition for the preparation of a drug for treating pain, inflammation, and/or fever, or alternatively, the composition for use in the treatment of pain, inflammation, and/or fever, or alternatively, a method for the treatment of pain, inflammation, and/or fever comprising the administration of said composition. One method in accordance with the present invention, a drug product is used for treatment of pain, inflammation, and/or fever. A method of treating a pediatric patient by administering the pharmaceutical composition described herein.

### BRIEF DESCRIPTION OF THE DRAWINGS

The invention may be more fully understood with reference to the accompanying drawing figures and the descriptions thereof. Modifications that would be recognized by those skilled in the art are considered a part of the present invention and within the scope of the appended claims.

FIG. 1 is a chart showing a comparative plasma absorption profile of ibuprofen salt compared to ibuprofen.

### DETAILED DESCRIPTION OF THE INVENTION

The object of the present invention is a pharmaceutical composition in aqueous solution for oral administration, characterized by including:
Ibuprofen sodium at a concentration between 1.5% (w/v) and 5% (w/v), expressed as the equivalent percentage of ibuprofen in acid form; and
A flavor masking system including povidone and sodium bicarbonate.

The authors of the present invention have developed a pharmaceutical composition of ibuprofen sodium in aqueous solution for oral administration, which, surprisingly, has excellent organoleptic properties, completely masking the bitter taste and burning sensation in the throat thanks to the combination of povidone and sodium bicarbonate. The composition developed is a translucent ready-to-use solution, especially suitable for pediatric application because it has the active ingredient in the adequate proportions to facilitate the proper administration in children, enabling the adjustment of doses according to body weight of each patient, plus excellent organoleptic qualities for acceptance by the pediatric patient.

Definitions

Throughout the present description, unless otherwise expressly stated, the proportions of the ingredients of the composition of the invention are expressed as weight/volume (w/v) percentages; for example, as grams of each component per 100 mL of the composition. Throughout the present description, unless otherwise expressly stated, the amounts of ibuprofen sodium are expressed as the equivalent amount of the drug in acidic form. A person skilled in the art will have no difficulty in converting values appropriately, based on the molecular weights of said substances. For example, when a solution of ibuprofen sodium is defined as 4% (w/v), this percentage is referring to the equivalent ibuprofen acid form, and actually correspond to a 4.43% (w/v) solution of ibuprofen sodium or to a 5.12% (w/v) solution of ibuprofen sodium dihydrate.

Throughout the present description, any number preceded by the term “about” indicates that the number also includes a variation of 5% above and below the stated value.

Ibuprofen Sodium

Ibuprofen sodium is the active ingredient of the composition. The meaning of the terms active ingredient, active principle, or drug substance is the substance that causes the pharmacological effect of the pharmaceutical preparation.

Ibuprofen is the International Non-proprietary Name (INN) by which (R,S)-2-(4-isobutylphenyl) propionic acid is commonly referred.

Therefore, ibuprofen sodium or ibuprofen sodium salt is (R,S)-2-(4-isobutylphenyl) propionate sodium.

Under the name ibuprofen sodium or ibuprofen sodium salt, their solvated forms, including hydrated forms, are commonly referred. In particular, it includes the dihydrate form or ibuprofen sodium dihydrate.

It is known that the active enantiomer of ibuprofen is the (S) form, although it has been found that mammals possess an isomerase capable of converting the (R) form into the active (S) form.

In the context of the present invention, ibuprofen used in the corresponding sodium salt includes ibuprofen in its racemic form ((R,S)-ibuprofen), the (S) enantiomer of ibuprofen ((S)-ibuprofen), and a mixture of the (R) and (S) enantiomers of ibuprofen, preferably enriched in the (S) form. Preferably, the sodium salt of ibuprofen used in the present invention is selected from the sodium salt of ibuprofen (R,S) and the sodium salt of ibuprofen (S). More preferably, the compositions of the invention contain the sodium salt of ibuprofen (R,S).

Ibuprofen sodium is commercially available or can be prepared, for example, as described in the patent application WO89/09053-A1, by reaction between ibuprofen in acid form with sodium bicarbonate.

Ibuprofen sodium in the composition of the invention is in a proportion between 1.5% (w/v) and 5% (w/v), more preferably between 1.7% (w/v) and 4.5% (w/v), and even more preferably between 1.9% (w/v) and 4.1% (w/v). Percentages of ibuprofen sodium are expressed as equivalent percentages of ibuprofen in acid form.

In a preferred embodiment of the invention, ibuprofen sodium is in a proportion of about 2% (w/v), expressed as the equivalent percentage of ibuprofen in acid form.

In another preferred embodiment of the invention, ibuprofen sodium is in a proportion about 4% (w/v), expressed as the equivalent percentage of ibuprofen in acid form.

Taste Masking System

The authors of the present invention have found, surprisingly, that the combination of the masking effect of chemical type facilitated by sodium bicarbonate, plus the masking effect of physical type favored by povidone completely eliminates the unpleasant bitter taste and the perceived feeling of irritation and burning in the throat caused by ibuprofen when is administered in soluble form. This optimal masking effect is accomplished even using relatively high concentrations of ibuprofen sodium, up to about 5%, which are especially suitable for pediatric administration.

One ingredient of the taste masking system is sodium bicarbonate, also known as sodium hydrogen carbonate, or sodium acid carbonate, whose formula is NaHCO₃. This ingredient is a salt whose hydrolysis provides basic pH solutions.

Sodium bicarbonate is generally added in an amount in line with the proportion of the active substance in the composition, so as to the weight ratio of sodium bicarbonate to ibuprofen sodium is generally between 1:1 and 1:5, preferably between 1:2 and 1:4, wherein the amount of ibuprofen sodium is expressed as the equivalent amount of ibuprofen in acid form.

Based on those ratios, a person skilled in the art will have no difficulty in calculating the preferable percentage of sodium bicarbonate to be used in the composition.

Thus, for example, in one of the preferred embodiments of the invention in which ibuprofen sodium is in a proportion of 2% (w/v), expressed as the equivalent percentage of ibuprofen in acid form, sodium bicarbonate is usually in a proportion between 0.4% (w/v) and 2.0% (w/v), preferably between 0.5% (w/v) and 1% (w/v). For another of the preferred embodiments of the invention in which ibuprofen sodium is in a proportion of 4% (w/v), expressed as the equivalent percentage of ibuprofen in acid form, sodium bicarbonate is generally in a proportion between 0.8% (w/v) and 4.0% (w/v), preferably between 1.0% (w/v) and 2.0% (w/v).

The second component of the flavor masking system is povidone, also known as polyvinylpyrrolidone or PVP, which is 1-vinyl-2-pyrrolidone homopolymer (CAS number 9003-39-8).

Povidone is commercially available from several distributors, such as BASF, under the trade name Kollidone.

Povidone may show different grades of polymerization, resulting in different molecular weights and, in turn, in different grades of viscosity in aqueous solution. These different grades of viscosity are quantified with the K value, as described, for example, in the following recognized manual of pharmaceutical excipients: R. C. Rowe et al. Handbook of Pharmaceutical Excipients. 4th edition, Pharmaceutical Press, London, 2003 [ISBN: 0-85369-472-9].

Any grade of povidone could be used. Preferably, povidone is selected from povidone with a K value between 17 and 90, more preferably from povidone K-17, povidone K-30 and povidone K-90, and even more preferably, povidone K-90 is used.

The composition of the invention contains an amount of povidone, so as to the weight ratio of povidone to ibuprofen sodium is generally between 1:0.4 and 1:8, preferably between 1:0.5 and 1:5, more preferably between 1: 0.7 and 1:4, and even more preferably between 1:1 and 1:2, wherein the amount of ibuprofen sodium is expressed as the equivalent amount of ibuprofen in acid form.

Based on those ratios, a person skilled in the art will have no difficulty in calculating the appropriate percentage of povidone used in the composition, depending on the percentage of the active ingredient.

Thus, for example, in one of the preferred embodiments of the invention according to which ibuprofen sodium is in a proportion of 2% (w/v), expressed as the equivalent percentage of ibuprofen in acid form, povidone is generally in an amount between 0.25% (w/v) and 5% (w/v), preferably between 0.4% (w/v) and 4% (w/v), more preferably between 0.5% (w/v) and 2.9% (w/v), and even more preferably between 1% (w/v) and 2% (w/v). For another preferred embodiments of the invention according to which ibuprofen sodium is in a proportion of 4% (w/v), expressed as the equivalent percentage of ibuprofen in acid form, povidone is generally in a proportion between 0.5% (w/v) and zo 10% (w/v), preferably between 0.8% (w/v) and 8% (w/v), more preferably between 1.0% (w/v) and 5.8% (w/v), and even more preferably between 2% (w/v) and 4% (w/v).

Notably, the proportion of sodium bicarbonate effective to mask the unpleasant bitter taste and the feeling of irritation and burning caused by ibuprofen sodium in a solution is significantly lower than that described in the prior state of the art as effective.

The pH of the resulting solution is generally between 7.5 and 8.5, a value which is suitable so that the preservatives p-hydroxybenzoates or parabens, that may be present in the composition, are stable and retain their antimicrobial activity. This will allow to obtain a liquid pharmaceutical preparation ready for use as the present invention, which can be preserved for microbiological stability throughout its life.

So, the authors have developed an ibuprofen sodium composition in which, surprisingly, the synergy generated by the two components of the masking system avoids the need of unacceptably high amounts that would be required of either ingredient for obtaining a similar masking effect. Also, that synergy avoids the need of extreme pH values at which the preservatives commonly used in oral fluids are inactive.

The surprising effect is that, according to the example 2 in the European patent application EP0418043-A, a preparation containing 512 mg of ibuprofen sodium, equivalent to 400 mg of ibuprofen plus 500 mg of sodium bicarbonate in 100 mL of water, was not acceptable in terms of the taste masking, since the burning sensation was still detectable. Only when the amount of sodium bicarbonate was increased to 700 mg, the objectionable taste and burning sensation associated with the sodium ibuprofen were substantially undetectable. That means that an overage of sodium bicarbonate, in relation to the active ingredient, is required in order to obtain an acceptable taste (700 mg of sodium bicarbonate vs 400 mg of ibuprofen or a 1.75:1 ratio). In the composition of the present invention, only 1.8 g of sodium bicarbonate were required in order to mask the objectionable taste and burning sensation produced by 5.12 g of ibuprofen sodium, equivalent to 4 g of ibuprofen, as shown in the Example 1. That means a 0.45:1 ratio, in a 10 times more concentrated solution than those described in the European patent application EP0418043-A.

The surprising taste masking effect described above, enables the possibility of formulating a preparation with two special features, which have not been reported in the state of the art:

First, the low content of sodium bicarbonate allows a preparation with low pH, between 7.5 and 8.5, at which the common pharmaceutical preservatives are effective, allowing us to obtain a multi-dose, ready-to-use, liquid preparation;

and second, the high concentration obtained, 10 times higher than the reported in the European patent application EP0418043-A, enables the possibility of a suitable product to be administered in children, dosed in small volumes according to the body weight of each pediatric patient.

Other Components

The composition of the invention is an aqueous solution, that is, the major component is water, wherein the sodium salt of ibuprofen, the masking system components, and, eventually, other additional optional components are dissolved.

Purified water is usually used, according to its specifications, for example, in the European Pharmacopoeia (Ph Eur) or United States Pharmacopeia (USP).

Besides water, the active ingredient, and the components of the taste masking system, the composition of the invention may optionally contain additional ingredients such as preservatives, cosolvents, sequestrants, sweeteners, flavors, crystallization inhibitors, or colorants.

The characteristics of some of the referred excipients are described, for example, in the manual written by Rowen et al. previously cited.

Preservative

The composition of the invention generally includes a preservative to ensure microbiological stability of the preparation, which, because of its high proportion of water, may be susceptible to microbiological contamination.

Any suitable preservative could be used to achieve the microbiological stability of pharmaceutical solutions for oral administration, as is well known to those skilled in pharmaceutical technology, and could be used in amounts which are also common as indicated, for example, in chapter 39 (Crowley M. M., Solutions, Emulsions, Suspensions and Extracts) of the recognized pharmaceutical technology manual Remington: The Science and Practice of Pharmacy, 21st Edition, Lippincott Williams & Wilkins, Philadelphia, 2005 [ISBN: 0-683-306472], or in Handbook of Preservatives, Michael and Irene Ash, Synapse Information Resources, 2004 [ISBN 1-890595-66-7].

So, among suitable preservatives to be incorporated in the compositions of the present invention are, for example, parabens, esters of p-hydroxybenzoic acid, particularly methylparaben, ethylparaben, propylparaben or butylparaben, cetylpyridinium chloride, chlorhexidine acetate, chloramine T, or mixtures thereof. Preferably, a paraben or a mixture of parabens is used, and more preferably a mixture of methylparaben and propylparaben.

The amount added of such preservatives is adjusted according to the function of the preservative or mixture of preservatives used, as is well known to those skilled in pharmaceutical technology.

For example, butylparaben is usually added at concentrations between 0.1% and 0.4%; methylparaben, ethylparaben, or propylparaben can be added at concentrations between 0.01% and 0.25%; cetylpyridinium chloride is added at concentrations generally ranging from 0.1% to 1.5%; chlorhexidine acetate is added at concentrations generally ranging from 0.01% to 0.5%; and chloramine T is added at concentrations generally ranging between 0.001% 0.1%. All percentages are expressed in weight/volume.

Cosolvent

The aqueous composition of the invention may optionally have a cosolvent that improves the solubilization of some low water-soluble ingredients which may be present in the composition, such as some preservatives.

Some of the suitable cosolvents to be used with the present invention are propylene glycol, sorbitol, ethanol, or mixtures thereof.

If a cosolvent is to be used, it is preferably chosen from propylene glycol, polyethylene glycol, and mixtures thereof, being propylene glycol the preferred cosolvent.

If a cosolvent is to be used, it is generally added at a concentration ranging from 2.0% (w/v) and 20% (w/v), preferably between 4.0% (w/v) and 10% (w/v).

Sequestering Agent

Optionally, the composition of the invention may include a sequestrant or chelating agent, which has a stabilizing function of the solution due to its ability to bind metal ions. By doing so, it prevents possible oxidation of the active ingredient catalyzed by such ions.

Among the suitable sequestering agents to be included in the composition of the invention are, for instance, ethylenediaminetetraacetic acid (EDTA) or edetic acid, disodium edetate, citric acid, fumaric acid, malic acid, or mixtures thereof. Preferably, disodium edetate is used.

The sequestering agent is added at a suitable concentration, as is well known to the skilled person in the area. For example, edetate disodium may be used at concentrations generally ranging from 0.05% to 0.2%; citric acid at concentrations generally ranging from 0.1% to 3.0%; edetic acid at concentrations generally ranging from 0.05% and 0.2%; fumaric acid at concentrations generally ranging from 0.1% to 3.0%; and malic acid at concentrations generally ranging from 0.1% to 3.0%. Percentages are expressed in weight/volume.

Sweetener

Moreover, the composition may contain a sweetening agent to improve its flavor. Among suitable sweeteners to be used in the present invention are saccharin, saccharose, sucralose, aspartame, acesulfame potassium, trehalose, cyclamate, and mixtures thereof, among others.

The expert in pharmaceutical technology will have no difficulty in selecting an appropriate amount of the flavoring substance or substances in the composition.

As an indication, saccharin, sucralose, aspartame, acesulfame potassium, and cyclamate are generally used at concentrations between 0.05% and 1.0%, whereas sucrose may be added at concentrations between 5% and 60%, preferably between 15% and 45%. Percentages are expressed in weight/volume.

Flavor

The composition of the invention may optionally have flavoring agents to improve the sensation in the mouth. These flavorings are, for example, essential oils of natural origin as peppermint, mint arvensis, curly mint, eucalyptus, lemon, or lime, among others, or mixtures thereof. The flavoring agent can also be of synthetic origin, such as strawberry, banana, pineapple, caramel, vanilla, cinnamon, apricot, among others. The preferred flavoring agents are those soluble in water.

The flavor or the mixture of flavors are usually used at concentrations ranging from 0.5% (w/v) to 3.0% (w/v).

Crystallization Inhibitor

Optionally, the composition of the invention may have a substance acting as crystallization inhibitor to prevent the formation of crystals of the active ingredient or any other component, around the mouth of the bottle containing the preparation.

Among the substances that can be used as crystallization inhibitors are sorbitol, xylitol, glycerin, and mixtures thereof. Preferably, sorbitol is used.

The crystallization inhibitor is generally added at a concentration between 1.0% and 30%, preferably between 3.0% and 20%, and more preferably between 5.0% and 10%. Percentages are expressed in weight volume.

Characteristics and Use of the Composition

According to the present invention, the composition is an ibuprofen sodium solution, translucent, slightly viscous, and with excellent organoleptic properties thanks to the combination of povidone and sodium bicarbonate. When the solution is put in the mouth, the bitter taste is not detected, and there is no irritation or burning sensation in the back of the throat when swallowed.

This excellent taste masking effect provided by the combination of povidone and sodium bicarbonate is achieved even in solutions with a relatively high content of the active ingredient, for example, up to 5%. These high concentrations are the key that enables the administration of the preparation in young children, in easy-to-use and adjustable volumes containing the exact required doses according to body weight of each pediatric patient.

The excellent organoleptic quality of the composition of the invention makes it especially adequate for pediatric patients, because these patients are particularly prone to reject a dosage form that is not organoleptically acceptable, resulting in a therapeutic problem. The composition developed, which contains dissolved ibuprofen sodium in a dissociated form, allows the absorption into the body up to three times faster as compared to the use of ibuprofen acid in suspension, as ibuprofen is commonly used in pediatric formulations available in the market.

This translates into a reduction in the time required to observe the therapeutic effects of reducing pain, inflammation, and/or fever in children.

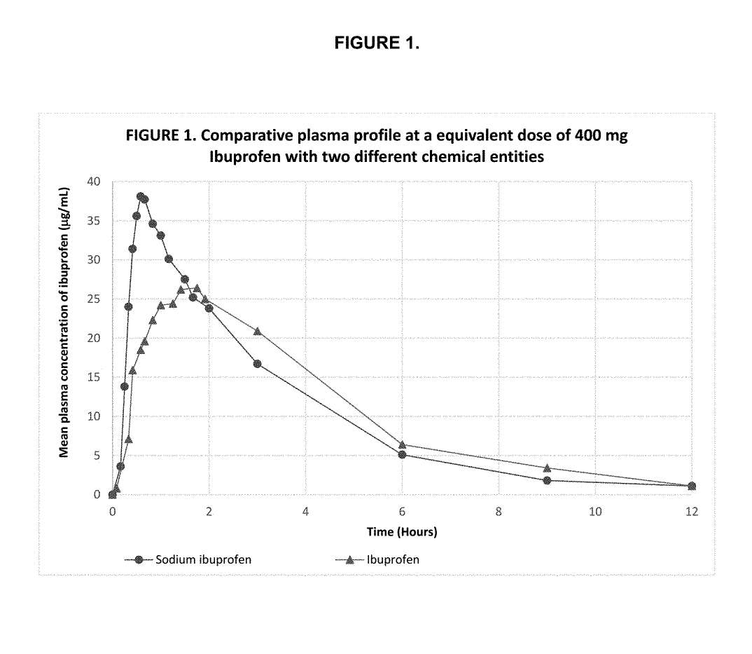

As described in FIG. 1, the evolution of ibuprofen plasma concentration-time in individuals who were given 10 mL of the composition of the invention prepared in the referred example, containing 512 mg of ibuprofen sodium dihydrate, equivalent to 400 mg of ibuprofen in acid form, was compared to that observed with the administration of an equivalent amount of the active ingredient, but administered as a suspension of ibuprofen in acid form.

It was found that the composition of the invention reaches higher plasma levels, with a Cₘₐₓ of 38 μg/mL, in comparison to a Cₘₐₓ of 26 μg/mL reached after the administration of an equivalent dose of ibuprofen in its acid form.

Additionally, it was observed that the time required to reach the peak plasma concentration (Tₘₐₓ) is significantly lower for ibuprofen sodium dihydrate, being about 35 minutes, while the Tₘₐₓ for the acid form is three times higher, 105 minutes.

Another aspect of the invention is the composition of the invention for use to treat pain, inflammation, and/or fever. In other words, another aspect of the invention is the use of the composition of the invention for the preparation of a drug product for the treatment of pain, inflammation, and/or fever. And expressed even in different words, another aspect of the invention is a method for the treatment of pain, inflammation, and/or fever in a patient in need, administering the composition of the invention.

The dose of the active ingredient administered may vary depending on the specific indication and the severity of the clinical condition, as well as the body weight of the pediatric patient.

Thus, for pediatric patients the recommended daily dose of ibuprofen is generally between 20 and 30 mg/kg, divided in three or four doses.

By way of guidance, the recommended adult dosage of ibuprofen ranges from 800 mg to 1,800 mg per day divided in three doses. It is recommended not to exceed 2,400 mg daily.

The composition of the invention is appropriate for the referred use or method of treatment in patients of any age, although its specific characteristics regarding dosage form, organoleptic qualities, and concentration of the active ingredient make it particularly adequate for administration to pediatric patients.

Thus, a preferred embodiment of the invention relates to such use or method of treatment in pediatric patients.

In the context of the present invention, the pediatric patient covers children from 3 months to 12 years of age, approximately.

The following is an example which is included for illustrative purposes of the present invention, and as such, it should not be understood as limiting the invention.

### EXAMPLES

### Example 1

### Aqueous Composition of Ibuprofen Sodium

| Component | % (w/v) | Function |
|---|---|---|
| Ibuprofen sodium | 5.12 | Active ingredient |
| dihydrate | | |
| Sodium bicarbonate | 1.80 | Masking agent |
| Povidone K 90 | 2.00 | Masking agent |
| Disodium edetate | 0.10 | Sequestrant |
| Sorbitol (70% solution) | 10.00 | Crystallization inhibitor |
| Propylene Glycol | 4.00 | Cosolvent |
| Methylparaben | 0.20 | Preservative |
| Propylparaben | 0.02 | Preservative |
| Saccharose (sucrose) | 30.00 | Sweetener |
| Sodium Saccharin | 0.60 | Sweetener |
| Sucralose | 0.30 | Sweetener |
| Caramel flavor | 0.80 | Flavoring |
| Red fruit flavor | 1.00 | Flavoring |
| Red color | 0.10 | Dye |
| Purified water | s.q.f. | Vehicle |
| | 100.00 | |

In order to prepare the composition, disodium edetate was dissolved in 50 mL of purified water, and then sodium bicarbonate and povidone were added under constant stirring. In a separate container, sucrose was dissolved in 35 mL of purified water under constant stirring and heating at 55° C., and then sorbitol was added and stirred until obtaining a homogeneous mixture. The solution thus obtained was added to the first solution prepared with disodium edetate, sodium bicarbonate, and povidone, and stirred until obtaining a homogeneous mixture.

To the above solution, ibuprofen sodium dihydrate, previously dissolved in 62.5 mL of purified water, was added.

In a separate container, methylparaben and propylparaben were dissolved in propylene glycol under constant stirring and heating at 55° C. After dissolution, they were added to the previous solution, and the solution was stirred until obtaining a homogeneous mixture.

To the previous solution, sodium saccharin, previously dissolved in 4 mL of purified water, sucralose previously dissolved in 4 mL of purified water, the red color previously dissolved in 2 mL of purified water, and flavors were added consecutively, stirring the solution after each addition until obtaining a homogeneous mixture.

Finally, a volume of 250 mL was made with purified water.

The composition obtained was a translucent, slightly viscous, red solution.

The composition was assessed from an organoleptic point of view by a panel of three people. The panelists agreed that the sensory perception in the mouth was slightly sweet and without a detectable bitter taste, and that it did not cause irritation or burning sensation in the back of the throat when swallowed.

Moreover, 10 mL of the composition were given to different volunteers, and ibuprofen plasma concentration was analyzed at regular time intervals of 12 hours. The dose administered was 512 mg of ibuprofen sodium dihydrate, equivalent to 400 mg of ibuprofen in acid form. The mean peak plasma concentration (Cₘₐₓ) was 38 μg/mL, reached in an average time of 35 minutes after administration (Tₘₐₓ).

On the other hand, 10 mL of a commercial ibuprofen composition in acid form were administered as a 4% suspension, containing 400 mg of ibuprofen, and the evolution of ibuprofen plasma concentration versus time was also analyzed. In this case, the mean peak plasma concentration (Cₘₐₓ) was 26 μg/mL, reached in an average time of 105 minutes after administration (Tₘₐₓ).

Therefore, it was evidenced with this comparative test that the composition of the invention containing sodium ibuprofen in dissociated form, reached a higher peak plasma concentration and also decreased the time required to reach the peak plasma concentration.

Although this invention has been described in connection with specific forms and embodiments thereof, it will be appreciated that various modifications other than those discussed above may be resorted to without departing from the spirit or scope of the invention. For example, equivalent elements may be substituted for those specifically shown and described, certain features may be used independently of other features, and in certain cases, particular locations of elements may be reversed or interposed, all without departing from the spirit or scope of the invention as defined in the appended claims.

1. A pharmaceutical composition, in aqueous solution, comprising:
ibuprofen sodium dihydrate in a concentration ranging from 1.92% (w/v) to 6.41% (w/v), equivalent to 1.5% (w/v) to 5.0% (w/v) of ibuprofen in acid form;
povidone; and
sodium bicarbonate; wherein the pH value of the aqueous solution is between 7.5 and 8.5.

2. The pharmaceutical composition of claim 1, wherein the ibuprofen sodium dihydrate concentration is 2.56% (w/v), equivalent to 2% (w/v) of ibuprofen in acid form.

3. The pharmaceutical composition of claim 1, wherein the ibuprofen sodium dihydrate concentration is 5.12% (w/v), equivalent to 4% (w/v) of ibuprofen in acid form.

4. The pharmaceutical composition of claim 1, wherein the weight ratio of sodium bicarbonate to ibuprofen is between 1:1 and 1:5.

5. The pharmaceutical composition of claim 1, wherein the povidone is selected from the group consisting of povidone K-17, povidone K-30, and povidone K-90.

6. The pharmaceutical composition of claim 1, wherein the weight ratio of povidone to ibuprofen is between 1:0.4 and 1:8.

7. (canceled)

8. The pharmaceutical composition of claim 1, further comprising a preservative agent selected from the group consisting of methylparaben, ethylparaben, propylparaben, butylparaben, cetylpyridinium chloride, chlorhexidine acetate, chloramine T, and mixtures thereof.

9. The pharmaceutical composition of claim 8, wherein the preservative is a mixture of methylparaben and propylparaben.

10. The pharmaceutical composition of claim 1, further comprising a cosolvent.

11. The pharmaceutical composition of claim 10, wherein the cosolvent selected from the group consisting of propylene glycol, polyethylene glycol, sorbitol, ethanol, and mixtures thereof.

12. The pharmaceutical composition of claim 10, wherein the cosolvent is propylene glycol.

13. The pharmaceutical composition of claim 1, further comprising a sequestering agent.

14. The pharmaceutical composition of claim 13, wherein the sequestering agent is selected from the group consisting of edetic acid, disodium edetate, citric acid, fumaric acid, malic acid, and mixtures thereof.

15. The pharmaceutical composition of claim 13, wherein the sequestering agent is disodium edetate.

16. The pharmaceutical composition of claim 1, further comprising at least one additional component selected from a sweetener, a flavor, and a crystallization inhibitor.

17. The pharmaceutical composition of claim 16, wherein the sweetener is selected from the group consisting of saccharin, saccharose, sucralose, aspartame, acesulfame potassium, trehalose, cyclamate, and mixtures thereof.

18. The pharmaceutical composition of claim 16, wherein the flavor is selected from the group consisting of essential oils of natural origin as peppermint, mint arvensis, curly mint, eucalyptus, lemon, or lime, or mixtures thereof.

19. A method of use of the pharmaceutical composition of claim 1, for preparing a drug product for treatment of pain, inflammation, and/or fever.

20. The method of claim 19, wherein the composition is used for treatment of pediatric patients.
